(19) United States
(12) Patent Application Publication
(10) Pub. No.: US 2021/0196598 A1
(43) Pub. Date: Jul. 1, 2021

(54) Specific Co-Aggregation Inhibition by Arginine

(71) Applicant: Colgate-Palmolive Company, New York, NY (US)

(72) Inventors: Carlo DAEP, Brooklyn, NY (US); Harsh Mahendra TRIVEDI, Hillsborough, NJ (US)

(73) Assignee: Colgate-Palmolive Company, New York, NY (US)

(21) Appl. No.: 16/649,298

(22) PCT Filed: Dec. 19, 2019
(86) PCT No.: PCT/US2019/067477

§ 371 Date: Mar. 20, 2020

Related U.S. Application Data
(60) Provisional application No. 62/785,115, filed on Dec. 26, 2018.

(51) Int. Cl. A61K 8/44; A61Q 11/00; C12Q 1/18

(52) U.S. Cl. CPC A61K 8/44; A61K 2800/92; C12Q 1/18; A61Q 11/00

(57) ABSTRACT

Methods of selectively inhibiting co-aggregation of commensal and pathogenic bacteria in a population of bacteria that comprises commensal and pathogenic bacteria, such as in an individual's oral cavity are disclosed. Methods of reducing biofilm in an individual's oral cavity are disclosed. Methods of inhibiting biofilm formation in a population of bacteria that comprises commensal and pathogenic bacteria are disclosed. Methods of identifying a compound or composition that enhances the selective inhibition of coaggregation of commensal and pathogenic bacteria by arginine or an arginine oligomer are also disclosed. Methods of identifying a compound or composition that enhances the selective inhibition of biofilm formation of commensal and pathogenic bacteria by arginine or an arginine oligomer are disclosed.

### BACKGROUND

Oral plaque is a highly complex biofilm that causes gingivitis and periodontitis. Oral plaque formation is a dynamic, stratified event. Formation of oral plaque requires coaggregation of oral microbes that helps to seed biofilm development. Primary colonizing bacteria such as Streptococcis (oralis group) and Actinomyces act as foundational bacteria that serve as the first colonizer of the oral plaque occupying the supragingival biofilms. Over time, gram negative facultative (Fusobacteria) and obligate anaerobes (Porphyromonads) interact with the supragingival microbes, obtaining important metabolic and environmental support under an oxidative environment until they can colonize a predominantly anaerobic subgingival environment.

The interaction between commensal bacteria and gram-negative microbes is transient as the latter group of organisms primarily resides within the subgingival pocket where conditions support the growth of these bacteria. It is also important to point out that Fusobacteria can reduce the local environment to facilitate the survival of anaerobes like Porphyromonads under aerobic conditions. The constant metabolic support between oral bacteria makes bacterial co-adhesion even more important. Furthermore, because constant sheer forces pose a physical challenge for bacteria colonizing the oral cavity, a tight cell-to-cell interaction or coaggregation between various colonizing bacteria may not only present a metabolic advantage over planktonic cells but directly facilitate the development of oral biofilms.

Co-aggregation of oral bacterial is thus a target for intervention for oral care products. Selectively inhibiting co-aggregation of commensal oral bacteria and pathogenic oral bacterial may provide a valid approach for promoting oral health. Such selective inhibition may be useful in methods of inhibiting biofilm formation, reducing biofilm pathogenicity and thereby preventing periodontitis and/or cavities/tooth decay. Methods of inhibiting co-aggregation of commensal oral bacteria and pathogenic oral bacterial in the oral cavity may provide a valid approach to change plaque composition towards a healthier state with less pathogens. Such method may promote good oral health and prevent or reduce the severity of diseases and disorders caused by pathogenic oral bacteria.

Methods to identify compounds and compositions that selectively inhibit co-aggregation of commensal oral bacteria and pathogenic oral bacterial in the oral cavity are also useful to identify compounds and compositions that prevent or inhibit biofilm formation, reduce biofilm pathogenicity and change plaque composition towards a healthier state with less pathogens. Thus, such methods are useful to identify compounds and compositions that promote good oral health and prevent or reduce the severity of diseases and disorders caused by pathogenic oral bacteria.

### BRIEF SUMMARY

Methods of preventing biofilm formation and reduction in biofilm pathogenicity are useful in changing plaque composition towards a healthier state with less pathogens.

Methods of selectively inhibiting co-aggregation of commensal and pathogenic bacteria within an individual's oral cavity are provided. The methods comprise the step of applying 1-50 mg of arginine or an arginine oligomer to the oral cavity.

Methods of selectively inhibiting co-aggregation of commensal and pathogenic bacteria in a population of bacteria that comprises commensal and pathogenic bacteria are provided. The methods comprise the step of: contacting the population of bacteria that comprises commensal and pathogenic bacteria with arginine or an arginine oligomer at a concentration of at least 40 nM.

Methods of reducing biofilm in an individual's oral cavity are provided. The methods comprise the step of applying 1-50 mg of arginine or an arginine oligomer to the oral cavity.

Methods of inhibiting biofilm formation in a population of bacteria that comprises commensal and pathogenic bacteria are provided. The methods comprise the step of: contacting the population of bacteria that comprises commensal and pathogenic bacteria with arginine or an arginine oligomer at a concentration of at least 40 nM.

Methods of identifying a compound or composition that enhances the selective inhibition of co-aggregation of commensal and pathogenic bacteria by arginine are provided. The methods comprise the steps of: performing a bacterial aggregation test assay and a bacterial aggregation control assay and comparing the results. The bacterial aggregation test assay comprises co-culturing at least one commensal bacteria and at least on pathogenic bacteria in media that comprises arginine and a test compound or composition and measuring bacterial aggregation by assigning a score using Visual Co-aggregation Scoring at one or more time points. The bacterial aggregation control assay comprises co-culturing at least one commensal bacteria and at least on pathogenic bacteria in media that comprises arginine and is free of a test compound or composition and measuring bacterial aggregation by assigning a score using Visual Co-aggregation Scoring at the one or more time points. The scores are compared and a reduced score in the bacterial aggregation test assay compared to the score in the bacterial aggregation control assay indicates that the test compound or composition enhances inhibition of bacterial aggregation by arginine.

Methods of identifying a compound or composition that enhances the selective inhibition of biofilm formation of commensal and pathogenic bacteria by arginine are provided. The methods comprise the steps of performing a biofilm formation test assay, performing a biofilm formation control assay, and comparing the results. The biofilm formation test assay comprises co-culturing at least one commensal bacteria and at least on pathogenic bacteria in media that comprises arginine and a test compound or composition and measuring biofilm formation at one or more time points. The biofilm formation control assay comprises co-culturing at least one commensal bacteria and at least on pathogenic bacteria in media that comprises arginine and is free of a test compound or composition and measuring biofilm formation at the one or more time points. The results of the biofilm formation test assay are compared to the results of the biofilm formation control assay. Reduced biofilm formation in the biofilm formation test assay compared to the biofilm formation in the biofilm formation control assay indicates that the test compound or composition enhances inhibition of biofilm formation by arginine.

### BRIEF DESCRIPTION OF THE DRAWINGS

FIG. 1 shows selective inhibition of commensal:pathogenic co-aggregation that is dose does dependent reduction in bacterial aggregate sizes (area based diameter, micrometer). The data in FIG. 1 shows reduction in bacterial aggregate following arginine treatments.

FIG. 2 shows normalization of the data from FIG. 1. The data in FIG. 2 shows arginine blocks commensal:pathogen co-aggregation.

### DETAILED DESCRIPTION

The following description of the preferred embodiment(s) is merely exemplary in nature and is in no way intended to limit the invention, its application, or uses.

As used herein, the expression “oral cavity” includes not only the cavity itself but also the saliva, teeth, gingiva, periodontal pockets, cheeks, tongue, mucosa, tonsils, any implants, and any device or structure which is placed into the oral cavity. The tonsils provide a reservoir (tonsil stones) for growth of anaerobic bacteria which may generate bad breath.

As used herein, arginine refers to the monomeric amino acid arginine. Arginine may be present as a free base or as a salt. In some embodiments, arginine refers to L-arginine.

As used herein, arginine oligomer refers to a 2-10 arginine oligomer, which is a short peptide having only arginine, i.e. a poly-arginine having 2-10 arginine monomers, i.e. arginine dimer (RR or R2), arginine trimer (RRR or R3), arginine tetramer (RRRR or R4), arginine pentamer (R5), arginine hexamer (R6), arginine heptamer (R7), arginine octamer (R8), arginine nonamer (R9) and arginine decamer (R10).

As used herein, the term “oral care composition” refers to a composition that is delivered to the oral surfaces. The composition may be a product which, during the normal course of usage, is not, the purpose of systemic administration of particular therapeutic agents, intentionally swallowed, but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, and the like.

In some embodiments, oral care compositions comprise arginine or an arginine oligomer. In some embodiments, such oral care compositions comprise arginine. In some embodiments, such oral care compositions comprise an arginine oligomer, i.e. R2, R3, R4, R5, R6, R7, R8, R9 or R10. In some embodiments, such oral care composition comprises two or more different arginine oligomers, i.e. two or more oligomers selected from R2, R3, R4, R5, R6, R7, R8, R9 and R10. In some embodiments, such oral care composition that comprises arginine and one or more different arginine oligomer, i.e. arginine plus one or more oligomers selected from R2, R3, R4, R5, R6, R7, R8, R9 and R10. Generally, compositions that comprise arginine include arginine and may or may not include one or more of R2-R10. Generally, compositions that comprise an arginine oligomer at least one of R2-R10 and may or may not comprise arginine and one or more of other R2-R10 arginine oligomers. In some embodiments, such oral care composition that comprises arginine monomer may optionally further comprise one or more or R2, R3, R4, R5, R6, R7, R8, R9 or R10. Likewise, in some embodiments, such oral care composition that comprises an arginine oligomer may comprise R2, R3, R4, R5, R6, R7, R8, R9 or R10 and optionally further comprise arginine and one or more additional arginine oligomers selected from R2, R3, R4, R5, R6, R7, R8, R9 and R10. An oral composition that comprises arginine or an arginine oligomer may comprise arginine and one or more arginine oligomers selected from R2, R3, R4, R5, R6, R7, R8, R9 and R10.

As used herein, the term “dentifrice” means paste, gel, or liquid formulations unless otherwise specified. In some embodiments, a dentifrice composition can be a combination of pastes, gels or paste and gel. In some embodiments, a dentifrice composition can be a combination of pastes, gels or paste and gel.

Arginine, a cationic amino acid, destabilizes oral biofilms (plaque) resulting in reductions in total biofilm biomass. No similar disruptive effects are observed among biofilms treated with another cationic amino acid, lysine.

Arginine can effectively block bacterial aggregation. The anti-coadhesion effect of arginine is selective; it is not equivalent among different bacterial groups. Arginine selectively inhibits co-aggregation of commensal and pathogenic bacteria. The anti-coadhesion effect of arginine on co-aggregation of commensal oral bacteria and pathogenic oral bacteria is greater that the anti-coadhesion effect of arginine on co-aggregation of commensal oral bacteria species. Selective inhibition of co-aggregation of commensal and pathogenic bacteria by arginine can reduce total biofilm and reduce overall biofilm pathogenicity, leading to a healthier mouth. Thus, arginine can be used in methods of reducing total biofilm in the oral cavity and in methods of reducing overall biofilm pathogenicity, leading to a healthier mouth. Reduction of total biofilm and overall biofilm pathogenicity in the oral cavity by arginine has the effect of changing plaque composition towards a healthier state with less pathogens.

Examples of commensal oral bacteria include Streptococcus gordonii, Actinomyces naeslundii and Actinomyces oris. The bacteria S. gordonii, A. naeslundii and A. oris are Gram-positive bacterium included among some of the initial colonizers of clean tooth surfaces and the periodontal environment.

Examples of pathogenic oral bacteria include Fusobacterium nucleatum, Porphyromonas gingivalis and Veillonella atypica. The bacteria F. nucleatum is a bacterium that is commonly found in the dental plaque of humans and is frequently associated with gum disease. It is a bacterium is a key component of periodontal plaque due to its abundance and its ability to coaggregate with other species in the oral cavity. The bacteria P. gingivalis is a pathogenic microbe associated with gum disease and malodor. The bacteria V. atypica are Gram-negative bacteria anaerobic cocci. Controlling the oral population of these bacteria would lead to improvement in oral health.

An example of arginine selectively blocking co-aggregation of commensal oral bacteria with pathogenic oral bacteria is the greater inhibition of co-aggregation of commensal oral bacteria A. naeslundii and pathogenic bacteria P. gingivalis compared to the level of inhibition of co-aggregation of commensal oral bacteria species S. gordonii and A. naeslundii. Another example of arginine selectively blocking co-aggregation of commensal oral bacteria with pathogenic oral bacteria is the greater inhibition of co-aggregation of commensal oral bacteria S. gordonii and pathogenic bacteria F. nucleatum compared to the level of inhibition of co-aggregation of commensal oral bacteria species S. gordonii and A. naeslundii. Other examples of arginine selectively blocking co-aggregation of commensal oral bacteria with pathogenic oral bacteria include greater inhibition of co-aggregation of any of the following commensal-pathogenic combinations: commensal oral bacteria S. gordonii and pathogenic bacteria V. atypica, commensal oral bacteria A. oris and pathogenic bacteria V. atypica, commensal oral bacteria A. oris and pathogenic bacteria F. nucleatum, and commensal oral bacteria A. oris and pathogenic bacteria P. gingivalis, compared to inhibition of co-aggregation of commensal oral bacteria species S. gordonii and A. oris. The inhibition of co-aggregation of bacteria results in an inhibition in biofilm formation.

Arginine or an arginine oligomer as a selective inhibitor of co-aggregation of commensal and pathogenic bacteria and inhibitor of biofilm formation may be provided to improve overall oral health. Arginine or an arginine oligomer may be provided in oral care compositions including, but not limited to, dentifrice and mouthrinse formulations. Oral care compositions that comprise arginine an arginine oligomer may be used to selectively inhibit co-aggregation of commensal and pathogenic bacteria in an individual's oral cavity. Oral care compositions that comprise arginine an arginine oligomer may be used to selectively inhibit, in an oral cavity, biofilm formation. Oral care compositions that comprise arginine an arginine oligomer may be used to reduce biofilm pathogenicity.

Methods are provided that selectively inhibit, in an oral cavity, co-aggregation of commensal and pathogenic bacteria. Methods are provided that selectively inhibit, in an oral cavity, biofilm formation by selective inhibition of co-aggregation of commensal and pathogenic bacteria. Methods are provided that selectively reduce, in an oral cavity, biofilm pathogenicity by selective inhibition of co-aggregation of commensal and pathogenic bacteria.

The methods provide the step of treating, applying or otherwise contacting the oral cavity of an individual with arginine or an arginine oligomer, preferably an oral care composition that comprises arginine or an arginine oligomer, such as for example a dentifrice that comprises arginine or an arginine oligomer, a toothpaste that comprises arginine or an arginine oligomer, a gel that comprises arginine or an arginine oligomer, a tooth powder that comprises arginine or an arginine oligomer, a mouthwash that comprises arginine or an arginine oligomer, a mouth rinse that comprises arginine or an arginine oligomer, a lozenge which may be dissolvable or chewable and which comprises arginine or an arginine oligomer, a tablet that comprises arginine or an arginine oligomer, a spray that comprises arginine or an arginine oligomer, a gum that comprises arginine or an arginine oligomer, or a film which may be wholly or partially dissolvable, or indissolvable and which comprises arginine or an arginine oligomer. In certain embodiments, the contacting of the oral cavity with the oral care composition that comprises arginine an arginine oligomer comprises the step of applying the oral care composition to the oral cavity using a brush, rinsing the oral cavity with the oral care composition in the form of a mouthwash, or spraying the oral care composition into the oral cavity using, for example, an atomizer. The individual or subject may be a mammal. In some embodiments, the individual or subject is a human. In some embodiments, the individual or subject is an animal, for example a companion animal (e.g. a cat or dog).

Arginine or an arginine oligomer is applied to the oral cavity of the individual in an effective amount of arginine or arginine oligomer. An effective amount of arginine or an arginine oligomer selectively inhibits co-aggregation of commensal and pathogenic bacteria, and/or inhibits biofilm formation and/or reduces biofilm pathogenicity. In some embodiments, the oral cavity of an individual is contacted with arginine or an arginine oligomer in an amount sufficient to provide arginine in the oral cavity at a concentration of at least 50 mM, preferably at least 75 mM, preferably at least 86 mM, preferably at least 100 mM or more, preferably at least 200 mM, preferably at least 300 mM or, more preferably at least 400 mM or more. In some embodiments, the oral cavity of an individual is contacted with arginine or an arginine oligomer in an amount sufficient to provide arginine or an arginine oligomer in the oral cavity at a concentration of at least 5 micromol/ml, in some embodiments at least 10 micromol/ml, in some embodiments at least 20 micromol/ml, in some embodiments at least 25 micromol/ml or more, in some embodiments about 20-25 micromol/ml, in some embodiments about 0.05-0.1 mg/ml, in some embodiments about 0.1-1.0 mg/ml or more, in some embodiments about 1.0-5.0 mg/ml, in some embodiments about 5-10 mg/ml, in some embodiments about 10-15 mg/ml, in some embodiments about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml or about 15 mg/ml. In some embodiments, the oral cavity of an individual is contacted with 0.1-50 mg of arginine or an arginine oligomer, in some embodiments, 0.25-50 mg of arginine or an arginine oligomer, in some embodiments, 0.5-50 mg of arginine or an arginine oligomer, in some embodiments, 1-50 mg of arginine or an arginine oligomer, in some embodiments, 5-50 mg, of arginine or an arginine oligomer in some embodiments, 10-40 mg of arginine or an arginine oligomer, in some embodiments, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg of, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of arginine or an arginine oligomer.

In some embodiments, arginine is applied to the oral cavity of the individual in an effective amount of arginine. An effective amount of arginine selectively inhibits co-aggregation of commensal and pathogenic bacteria, and/or inhibits biofilm formation and/or reduces biofilm pathogenicity. In some embodiments, the oral cavity of an individual is contacted with arginine in an amount sufficient to provide arginine in the oral cavity at a concentration of at least 50 mM, preferably at least 75 mM, preferably at least 86 mM, preferably at least 100 mM or more, preferably at least 200 mM, preferably at least 300 mM or, more preferably at least 400 mM or more. In some embodiments, the oral cavity of an individual is contacted with arginine in an amount sufficient to provide arginine in the oral cavity at a concentration of at least 5 micromol/ml, in some embodiments at least 10 micromol/ml, in some embodiments at least 20 micromol/ml, in some embodiments at least 25 micromol/ml or more, in some embodiments about 20-25 micromol/ml, in some embodiments about 0.05-0.1 mg/ml, in some embodiments about 0.1-1.0 mg/ml or more, in some embodiments about 1.0-5.0 mg/ml, in some embodiments about 5-10 mg/ml, in some embodiments about 10-15 mg/ml, in some embodiments about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml or about 15 mg/ml. In some embodiments, the oral cavity of an individual is contacted with 0.1-50 mg of arginine, in some embodiments, 0.25-50 mg of arginine, in some embodiments, 0.5-50 mg of arginine, in some embodiments, 1-50 mg of arginine, in some embodiments, 5-50 mg, of arginine in some embodiments, 10-40 mg of arginine, in some embodiments, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg of, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg of arginine.

The methods provided herein that selectively inhibit, in an oral cavity, co-aggregation of commensal and pathogenic bacteria and/or that inhibit, in an oral cavity, biofilm formation and/or reduce, in an oral cavity, biofilm pathogenicity have the effect of changing plaque composition towards a healthier state with less pathogens. The methods provided herein that selectively inhibit, in an oral cavity, co-aggregation of commensal and pathogenic bacteria may be useful as methods of preventing one or more of gingivitis, periodontitis, peri-implantitis, peri-implant mucositis, necrotizing gingivitis, necrotizing periodontitis and caries.

In some embodiments, one or more pathogenic oral bacteria species selected from the group consisting of: Fusobacterium nucleatum, Porphyromonas gingivalis and Veillonella atypica is identified as being present in the oral cavity of the individual.

In some embodiments, one or more commensal oral bacteria species selected from the group consisting of: Streptococcus gordonii, Actinomyces naeslundii and Actinomyces oris. is identified as being present in the oral cavity of the individual.

Arginine has been found to have a greater effect promoting co-aggregation of commensal bacteria with other commensal bacteria or co-aggregation of commensal bacteria with pathogenic bacteria growth compared to co-aggregation of pathogenic bacteria with other pathogenic bacteria. Arginine and arginine oligomers may be used in methods of reducing total pathogenic oral bacteria load within the oral cavity. Arginine and arginine oligomers may be used in methods of selectively inhibiting co-aggregation of commensal and pathogenic bacteria within an individual's oral cavity. Arginine and arginine oligomers may be used in methods of reducing biofilm in an individual's oral cavity. Arginine and arginine oligomers may be useful to changing plaque composition towards a healthier state with less pathogens.

Methods may comprise the step of treating or supplementing the oral cavity of an individual by applying or contacting the oral cavity of the individual with an effective amount of arginine or arginine oligomer, preferably an oral care composition that comprises arginine or arginine oligomer, such as for example a dentifrice that comprises arginine or arginine oligomer, a toothpaste that comprises arginine or arginine oligomer, a gel that comprises arginine, a tooth powder that comprises arginine or arginine oligomer, a mouthwash that comprises arginine or arginine oligomer, a mouth rinse that comprises arginine or arginine oligomer, a lozenge which may be dissolvable or chewable and which comprises arginine or arginine oligomer, a tablet that comprises arginine or arginine oligomer, a spray that comprises arginine or arginine oligomer, a gum that comprises arginine or arginine oligomer, or a film which may be wholly or partially dissolvable, or indissolvable and which comprises arginine or arginine oligomer. In certain embodiments, the contacting of the oral cavity with the oral care composition that comprises arginine or arginine oligomer comprises the step of applying the oral care composition to the oral cavity using a brush, rinsing the oral cavity with the oral care composition in the form of a mouthwash, or spraying the oral care composition into the oral cavity using, for example, an atomizer. The individual or subject may be a mammal. In some embodiments, the individual or subject is a human. In some embodiments, the individual or subject is an animal, for example a companion animal (e.g. a cat or dog).

The methods provided herein that may be useful as methods of preventing one or more of gingivitis, periodontitis, peri-implantitis, peri-implant mucositis, necrotizing gingivitis, necrotizing periodontitis and caries.

In some embodiments, the methods selectively promote biofilm formation by beneficial oral bacteria, relative to biofilm formation by pathogenic oral bacteria, after 48 hours incubation with the beneficial oral bacteria and the pathogenic oral bacteria.

Some embodiments provide methods that comprise applying to the oral cavity of an individual an effective amount of arginine or arginine oligomer. Oral compositions which comprise arginine or arginine oligomer are used to apply the arginine or arginine oligomer to the oral cavity. In some embodiments, the oral compositions comprise arginine or arginine oligomer in combination with a source of zinc ions. In some embodiments, the oral compositions comprise arginine or arginine oligomer in combination with a source of zinc ions and a source of fluoride ions. The source of zinc ions may include zinc oxide particles such as zinc oxide particles that are from 1 to 7 microns. The source of zinc ions may be selected from zinc citrate, zinc sulfate, zinc silicate, zinc lactate, zinc phosphate, zinc oxide, or a combination thereof. The source of zinc ions may be a combination of zinc oxide and zinc citrate. The source of fluoride may be stannous fluoride.

Oral care compositions comprise arginine or a salt thereof. In some embodiments, the arginine or arginine oligomer in combination with is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 1% in the case of a mouthwash.

In some embodiments, the arginine oligomer is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine oligomer is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine oligomer is calculated as free form. In some embodiments, the arginine oligomer is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine oligomer is present in an amount of up to 5% by weight, further optionally from 0.5 to 5% by weight, still further optionally from 2.5 to 4.5% by weight, based on the total weight of the oral care composition. In some embodiments, arginine oligomer is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine oligomer is present in a dentifrice, at for example about 0.5-2 wt. %, e.g., and about 1% in the case of a mouthwash.

In some embodiments the oral care compositions comprise arginine or arginine oligomer in combination with ZnO. Such oral care compositions may optionally further comprise a fluoride source. In some embodiments, the ZnO particles may have an average particle size of from 1 to 7 microns. In some embodiments, the ZnO particles have an average particle size of 5 microns or less. In some embodiments, in addition to zinc oxide the composition may comprise other metal oxides as well such as stannous oxide, titanium oxide, calcium oxide, copper oxide and iron oxide or a mixture thereof. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

In some embodiments the oral care compositions comprise arginine or arginine oligomer in combination with a zinc ion source is selected from zinc oxide, zinc citrate, zinc lactate, zinc chloride, zinc acetate, zinc gluconate, zinc glycinate, zinc sulphate, sodium zinc citrate, zinc silicate, zinc phosphate, and combinations thereof. Such oral care compositions may optionally further comprise a fluoride source. Examples of effective amount of zinc ions is an amount of zinc effective inhibit erosion, e.g., from 0.005-5% zinc, e.g., 0.01-0.05% for a mouthwash or 0.1 to 3% for a dentifrice, e.g., a dentifrice comprising 1-3% zinc citrate.

In some embodiments the oral care compositions comprise arginine or arginine oligomer in combination with two or more zinc salts wherein at least one is zinc oxide and at least one is zinc citrate . . . Such oral care compositions may optionally further comprise a fluoride source. In some embodiments, the weight ratio or zinc oxide to zinc citrate is 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine or arginine oligomer to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weigh % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

One or more fluoride ion sources are optionally present in an amount providing a clinically efficacious amount of soluble fluoride ion to the oral care composition. A fluoride ion source is useful, for example, as an anti-caries agent. Any orally acceptable particulated fluoride ion source can be used, including stannous fluoride, sodium fluoride, potassium fluoride, potassium monofluorophosphate, sodium monofluoropho sphate, ammonium monofluorophosphate, sodium fluorosilicate, ammonium fluorosilicate, indium fluoride, amine fluoride such as olaflur (N′-octadecyltrimethylendiamine-N,N,N′-tris(2-ethanol)-dihydrofluoride), ammonium fluoride, titanium fluoride, hexafluorosulfate, and combinations thereof. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm)., product. In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. In some embodiments, the fluoride source is sodium fluoride which provides fluoride in an amount from 750-2000 ppm (e.g., about 1450 ppm). In some embodiments, the fluoride source is selected from sodium fluoride and sodium monofluorophosphate and which provides fluoride in an amount from 1000 ppm-1500 ppm. In some embodiments, the fluoride source is sodium fluoride or sodium monofluorophosphate and which provides fluoride in an amount of about 1450 ppm. In some embodiments, stannous fluoride is the only fluoride source. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts. In some embodiment, the fluoride source is a fluoride salt present in an amount of 0.1 wt. % to 2 wt. % (0.1 wt %-0.6 wt. %) of the total composition weight (e.g., sodium fluoride (e.g., about 0.32 wt. %) or sodium monofluorophosphate). e.g., 0.3-0.4%, e.g., ca. 0.32% sodium fluoride

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminum oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high-water absorption, e.g., at least 25 g/100 g, e.g. 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 1 14 marketed by J. M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g. about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3 wt. %, 4% wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g. about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g. silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate-light (e.g., about 10% precipitated calcium carbonate-light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3%.by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean-and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or wanning effects. Such ingredients illustratively include menthol, carvone, methyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprise an agent that interferes with or prevents bacterial attachment, e.g., ethyl lauroyl arginiate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g 1.5% wt. by total weight of the composition. In some embodiments, the oral composition include an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2% cocamidopropylbetaine. Some embodiments comprise a nonionic surfactant in an amount of from 0.5-5%, e.g, 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000, One or more PEGs are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGs) and mixtures thereof. Most humectants also function as sweeteners. In some embodiments, compositions comprise 15% to 70% or 30% to 65% by weight humectant. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates. Soluble pyrophosphates are useful anticalculus agents. The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g. about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139(M.W. 500,000), AN 1 19 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients include vitamins, minerals, amino acids, and mixtures thereof. Vitamins include Vitamins C and D, miamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy ani sole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophoshpate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, Methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %., or 0.2 to 0.7 wt %, or from 0.3 to 0.6 wt %, or from 0.4 to 0.5 wt %, e.g. about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, and 35% or more of water.

Methods of identifying a compound or composition that enhances the selective inhibition of co-aggregation of commensal and pathogenic bacteria by arginine or arginine oligomer comprise performing a bacterial aggregation test assay, performing a bacterial aggregation control assay and comparing the results. The bacterial aggregation test assay comprises co-culturing at least one commensal bacteria and at least on pathogenic bacteria in media that comprises arginine or arginine oligomer and a test compound or composition and measuring bacterial aggregation by assigning a score using Visual Co-aggregation Scoring at one or more time points. The bacterial aggregation control assay comprises co-culturing at least one commensal bacteria and at least on pathogenic bacteria in media that comprises arginine or arginine oligomer and is free of a test compound or composition and measuring bacterial aggregation by assigning a score using Visual Co-aggregation Scoring at the one or more time points. The score of the bacterial aggregation test assay is compared to to the score of the bacterial aggregation control assay. A reduced score in the bacterial aggregation test assay compared to the score in the bacterial aggregation control assay indicates that the test compound or composition enhances inhibition of bacterial aggregation by arginine or arginine oligomer.

Method of identifying a compound or composition that enhances the selective inhibition of biofilm formation of commensal and pathogenic bacteria by arginine or arginine oligomer, are also provided. The methods comprise performing a biofilm formation test assay, performing a biofilm formation control assay and comparing the results. The biofilm formation test assay comprises co-culturing at least one commensal bacteria and at least on pathogenic bacteria in media that comprises arginine or arginine oligomer and a test compound or composition and measuring biofilm formation at one or more time points. The biofilm formation control assay comprises co-culturing at least one commensal bacteria and at least on pathogenic bacteria in media that comprises arginine or arginine oligomer and is free of a test compound or composition and measuring biofilm formation at the one or more time points. The biofilm formation in the biofilm formation test assay is compared to the biofilm formation in the biofilm formation control assay. A reduction in biofilm formation in the biofilm formation test assay compared to the biofilm formation in the biofilm formation control assay indicates that the test compound or composition enhances inhibition of biofilm formation by arginine or arginine oligomer.

In methods of identifying compounds that enhance arginine's or arginine oligomer's inhibition of co-aggregation and biofilm formation, test assays and control are performed using the same one or more pairs of commensal and pathogenic bacteria. In some embodiments, commensal and pathogenic bacteria pairs are selected from the group consisting of: A. naeslundii and P. gingivalis, S. gordonii and F. nucleatum, S. gordonii and V. atypica, A. oris and V. atypica, and A. oris and F. nucleatum, and commensal oral bacteria A. oris and pathogenic bacteria P. gingivalis. In some embodiments, multiple test assays are performed using various pairings of commensal and pathogenic bacteria. In some embodiments, multiple test assays are performed for commensal and pathogenic bacteria pairs using various concentrations of test compound/composition. Some embodiments include a negative control in which pairs of the same commensal and pathogenic bacteria are cultured in media that does not contain test compound/composition. In such embodiments, bacterial aggregation is also measured in the negative control. A reduction in bacterial aggregation in a test assay compared to bacterial aggregation in the negative control assay indicated that the test compound/composition inhibits co-aggregation of the commensal and pathogenic bacteria pair. Some embodiments include a commensal:commensal bacteria pair that are also cultured in the presence of a test compound. In such embodiments, bacterial aggregation is also measured for commensal:commensal bacteria pair. In some embodiments, commensal:commensal bacteria pairs are selected from the group consisting of: S. gordonii and A. naeslundii, and S. gordonii and A. oris. Assays may be done using multiple commensal:commensal bacteria pairs. For each pair, multiple concentrations of test compound/composition may be used, generally the same concentrations used in the test assays done using pairs of commensal and pathogenic bacteria. A greater reduction in bacterial aggregation in a test assay using pairs of commensal and pathogenic bacteria compared to bacterial aggregation in assays using a commensal:commensal bacteria pair indicates that the test compound/composition selectively inhibits co-aggregation of the commensal and pathogenic bacteria pair compared to commensal:commensal bacteria. In some embodiments, the test compound/composition is a test composition that comprises arginine or arginine oligomer and one or more other compounds to be tested in combination with arginine or arginine oligomer. When testing a test composition that comprises arginine or arginine oligomer and one or more other compounds to be tested in combination with arginine or arginine oligomer, the method is a method of identifying compounds and compositions which modulate the selective inhibition of co-aggregation of commensal and pathogenic bacteria by arginine or arginine oligomer. In some embodiments, the amount of arginine or arginine oligomer present in the bacterial aggregation test assay is at least 40 mM, or at least 86 mM, or at least 100 mM, or at least 200 mM, or at least 300 mM, or at least 400 mM. In some embodiments, test assays include a series of test assays using the media supplemented with 5, 10, 20, and 25 micromol/ml of arginine or arginine oligomer. In some embodiments, the test assays are performed using a 96-well polystyrene microtiter plate. The bacterial cultures are incubated under conditions to which promote growth. In some embodiments, the bacterial cultures are incubated under anaerobic conditions or an environment containing 5% carbon dioxide. Bacterial growth and aggregation are monitored at time points, such as for example at 24 and 48 hours. In some embodiments, bacterial aggregation is measured using Visual Co-aggregation Scoring in which the coaggregation is visually assessed and assigned a score such as 0-4. In some embodiments, total biofilm is assessed by measuring the diameter of bacterial biofilm formed.

Biofilm can be grown on different surfaces such as poly-D-lysine, collagen-coated or Hydroxyapatite (HAP) disks. In some embodiments, biofilm is grown on the surface of a 35 mm×0.17 mm poly-D-lysine plate. In some embodiments, biofilm is grown on the surface of a 35 mm×0.17 mm collagen-coated plates. In some embodiments, biofilm is grown on 12 mm HAP disks. In some embodiments, biofilm is grown on 0.5″×0.04-0.06 HAP disks. In some embodiments, saliva, which contains oral bacteria, is collected and incubated with a substrate (e.g. poly-D-lysine, collagen-coated or HAP disks).

The methods are useful to identify compounds and compositions which inhibit biofilm formation and which result in less pathogenic biofilm in the oral cavity. The methods are useful to identify compounds and compositions that change plaque composition towards a healthier state with less pathogenic bacteria, thereby producing less damaging plaque. The methods are useful to identify compounds and compositions which enhance the effect of arginine or arginine oligomer which inhibits biofilm formation and which results in less pathogenic biofilm in the oral cavity. The methods are useful to identify compounds and compositions which enhance the effect of arginine or arginine oligomer to change plaque composition towards a healthier state with less pathogenic bacteria, thereby producing less damaging plaque.

### EXAMPLES

### Example 1

Coaggregation was studied using FlowCam® technology (Fluid Imaging Technologies, ME, USA). S. gordonii DL1, A. oris MG1, P. gingivalis W83, and F. nucleatum ATCC 25586 were harvested from batch cultures and washed once in coaggregation buffer by centrifuging for 10 mins at 3,000 g. After centrifugation, the pellets were re-suspended in coaggregation buffer and adjusted to an optical density of 1.0 (±0.1) at 600 nm. The cell suspensions were then subject to a 5-fold dilution in coaggregation buffer. Each pair was then loaded onto the FlowCam™ device and allowed to sit for one minute. The pairs were subsequently pumped through the device until they reached the flow cell. Data collection was initiated once the Olympus UPlanFLN 10X/0.30 objective was focused on the flowing particles. The FlowCam™ was run for 45 seconds at a flow rate of 0.3 ml/min and images were acquired at 10 frames per second. Flash duration was set to 8 μSec and the particle sizes were measured using area based diameter (ABD) with a particle filter set to detect between 5 and 10000 μm. The same procedure was tested for each pair while suspended in coaggregation buffer supplemented with various concentrations of arginine or lysine.

### Example 2

The following three pairs of co-aggregates were co-cultured and treated with either buffer (untreated; control) 86 mM arginine or 100 mM arginine:

1) Commensal oral bacteria Streptococcus gordonii and Actinomyces naeslundii,

2) Commensal oral bacterium A. naeslundii and pathogenic bacterium Porphyromonas gingivalis,

3) Commensal oral bacterium S. gordonii and pathogenic bacterium Fusobacterium nucleatum.

Data not only show that arginine can effectively block bacterial co-aggregation but also illustrate differences in anti-adhesion efficacies between inter-commensal and commensal-pathogen co-aggregation.

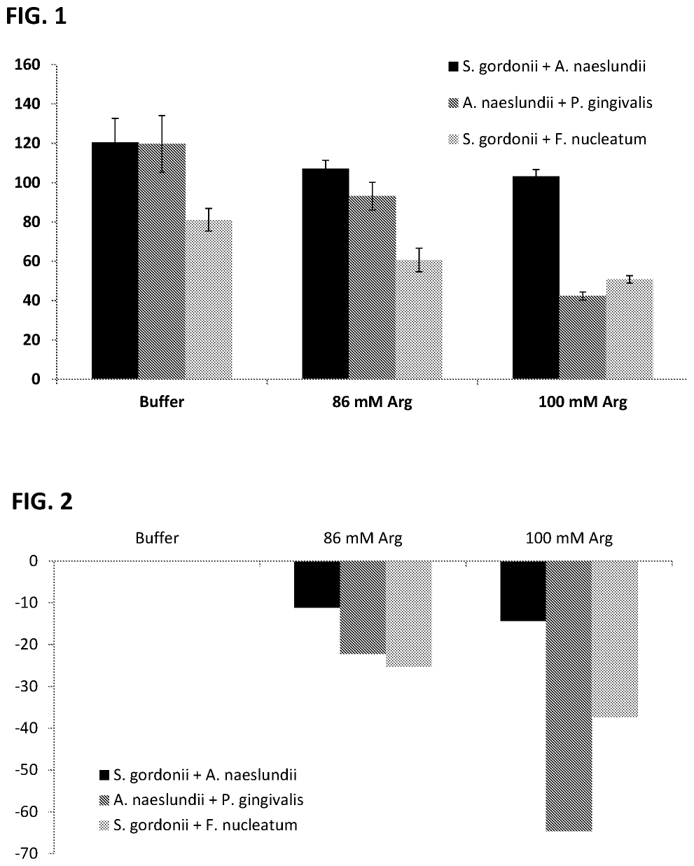

Data in FIG. 1 shows that treatment with 86 mM and 100 mM arginine showed dose dependent reduction in bacterial aggregate sizes (area based diameter, micrometer). The reduction in aggregate sizes was greater in each pair of commensal/pathogenic bacteria, i.e. the co-aggregates of A. naeslundii (commensal) and P. gingivalis (pathogenic) and the co-aggregates of S. gordonii (commensal) and F. nucleatum (pathogenic) compared to the commensal/commensal co-aggregates of S. gordonii (commensal) and A. naeslundii (commensal).

FIG. 2 shows normalization of the data from the experiments that generated the data shown in FIG. 1. In FIG. 2, the data from the treated co-aggregates was normalized against untreated co-aggregates. FIG. 2 reflects a selective action of arginine against specific co-aggregates. There was greater inhibition of co-aggregation by arginine in commensal-pathogenic co-aggregates compared to the level of inhibition of co-aggregation by arginine in the commensal:commensal co-aggregate. The commensal:commensal co-aggregate (S. gordonii and A. naeulundii) showed minimal reduction, inhibiting the interaction by only 11 and 14% after treatment with 86 mM and 100 mM arginine, respectively. Treating commensal:pathogenic co-aggregates with 86 mM and 100 mM arginine led to greater inhibitory function, blocking S. gordonii: F. nucleatum by 25% and 37%, respectively, and A. naeslundii: P. gingivalis by 22% and 64%, respectively. The greatest inhibitor activity was observed between A. naeslundii and P. gingivalis. This is consistent with the known transient interaction of the periodontal pathogenic bacterium with the commensal bacterium. Treatment with arginine may thus limit the biofilm colonization of P. gingivalis.

### Example 3

The following six pairs of co-aggregates were co-cultured and treated with either 86 mM arginine, 400 mM arginine, 86 mM lysine or 400 mM lysine:

1) Commensal oral bacteria S. gordonii and A. oris;

2) Commensal oral bacterium S. gordonii and pathogenic oral bacterium V. atypica;

3) Commensal oral bacterium S. gordonii and pathogenic oral bacterium F. nucleatum;

4) Commensal oral bacterium A. oris and pathogenic oral bacterium V. atypica;

5) Commensal oral bacterium A. oris and pathogenic oral bacterium F. nucleatum; and

6) Commensal oral bacterium A. oris and pathogenic oral bacterium P. gingivalis.

Bacterial aggregation was scored visually using a Visual Coaggregation Score (0-4) scoring system.

Data in Table 1 show that treatment with 86 mM and 100 mM arginine selectively inhibited aggregation in each pair of commensal/pathogenic bacteria, i.e. the commensal/pathogenic co-aggregate pairs S. gordonii and V. atypica, S. gordonii and F. nucleatum, A. oris and V. atypica, A. oris and F. nucleatum, and A. oris and P. gingivalis while having none to little inhibitory effect on the commensal/commensal co-aggregate pair S. gordonii and A. oris. Little to no anti-coadhesion functionality was observed for lysine in any of the corresponding pairs.

TABLE 1
Reversible Treatments of bacterial aggregation post-treatment
with amino acids Visual Coaggregation Score (0-4)

| Coaggregation Pairs | | | | |
|---|---|---|---|---|
| (selected pairs with | Arg | Arg | Lys | Lys |
| VSCs of 3-4) | 86 mM | 400 mM | 86 mM | 400 mM |
| S. gorndonii + A. oris | 4 | 3-4 | 4 | 4 |
| S. gorndonii + V. atypica | 2-3 | 2 | 4 | 3 |
| S. gorndonii + F. nucleatum | 0-1 | 0-1 | 3-4 | 3 |
| A. oris + V. atypica | 0-1 | 0 | 3 | 0-1 |
| A. oris + F. nucleatum | 0-1 | 0 | 3-4 | 2-3 |
| A. oris + P. gingivalis | 0-1 | 0 | 1-2 | 0-1 |

0 = no aggregates
4 = strong aggregates

### Example 4

Oral compositions that comprise arginine are disclosed in WO/2011/123123, which corresponds to U.S. Pat. No. 8,652,495, which are both incorporated herein by reference. In some embodiments, the oral care composition comprises an orally acceptable vehicle; zinc oxide particles; and arginine. In some such embodiments, zinc oxide particles have a median particle size of from 1 to 7 microns; in some such embodiments, the zinc oxide particles have a particle size distribution of 3 to 4 microns, a particle size distribution of 5 to 7 microns, a particle size distribution of 3 to 5 microns, a particle size distribution of 2 to 5 microns, or a particle size distribution of 2 to 4 microns. In some of these variously described embodiments, the zinc oxide particles are present in an amount of up to 5% by weight, such as from 0.5 to 2% by weight based on the total weight of the oral care composition. In some of these variously described embodiments, the zinc oxide particles the source of zinc oxide particles is selected from a powder, a nanoparticle solution; a nanoparticle suspension; a capsule; and a bead. In some of these variously described embodiments, the oral care composition further comprises at least one additional metal oxide selected from stannous oxide, titanium oxide, calcium oxide, copper oxide and iron oxide or a mixture thereof. In some of these variously described embodiments, the arginine is L-arginine. In some of these variously described embodiments, the arginine is present in an amount of up to 5% by weight, such as from 0.5 to 5% by weight, such 2.5 to 4.5% by weight as based on the total weight of the oral care composition. In some of the various embodiments, the oral composition further comprises in addition to arginine, at least one or more amino acids is selected from cysteine, leucine, isoleucine, lysine, L-lysine, alanine, asparagine, aspartate, phenylalanine, glutamate, glutamic acid, threonine, glutamine, tryptophan, glycine, valine, proline, serine, tyrosine, and histidine, and a combination of two or more thereof. In some of these variously described embodiments, the oral care composition further comprises a polymeric adherent material. In some of these variously described embodiments, the source of zinc oxide particles is a capsule, and the capsule comprises a polymeric adherent material and in some such embodiments, the polymeric adherent material comprises one or more cellulose polymers, such as embodiments in which at least one of said one or more cellulose polymers is a hydroxyalkyl cellulose polymer selected from hydroxypropylmethyl cellulose (HPMC), hydroxyethylpropyl cellulose (HEPC), hydroxybutylmethyl cellulose (HBMC), and carboxymethyl cellulose (CMC). In some of these variously described embodiments, the polymeric adherent material comprises a mixture of two hydroxyalkyl cellulose polymers having different molecular weights and the zinc oxide which is encapsulated in the mixture of two hydroxyalkyl cellulose polymers. In some of these variously described embodiments, the polymeric adherent material comprises one or more polymers selected from a poly (ethylene oxide) polymer, polyvinylpyrrolidone (PVP), polyethylene glycol (PEG)/polypropylene glycol (PPG) copolymer, ethylene oxide (EO)-propylene oxide (PO) block copolymers, ester gum, shellac, pressure sensitive silicone adhesives, methacrylates, or mixtures thereof. In some of these variously described embodiments, the oral care composition is a dentifrice composition, such as for example, a toothpaste or a gel. In some of these variously described embodiments, the oral care composition is formulated into a form selected from a mouth rinse, a gum, a dissolvable lozenge, and a dissolvable film.

### Example 5

Oral compositions that comprise arginine are disclosed in WO 2014/088572, which corresponds to US 2015/0313813, which are both incorporated herein by reference. In some embodiments the oral care composition comprises: from about 0.05 to about 5% by weight, of a zinc ion source; a fluoride ion source in an amount effective to deliver from about 500 to about 5,000 ppm fluoride, and from about 0.1 to about 10%, by weight, of arginine. In some such embodiments, the zinc ion source is selected from zinc citrate, zinc sulfate, zinc silicate, zinc lactate, zinc phosphate, zinc oxide, and combinations thereof, for example, in an amount effective to inhibit erosion. In some such embodiments, the oral composition is in the form of a dentifrice comprising an abrasive. In some such embodiments, the amount of zinc is 0.5 to 4% by weight. In some such embodiments, the compositions may further comprise one or more abrasives, one or more humectants, and one or more surfactants. In some such embodiments, the compositions may further comprise an effective amount of one or more alkali phosphate salts and/or an effective amount of one or more antibacterial agents and/or a whitening agent. In some such embodiments, the composition comprises zinc phosphate and one or more other sources of zinc ion. In some such embodiments, the pH of the composition is basic. In some such embodiments, the composition may comprise, in a silica abrasive dentifrice base: 1 to 3% zinc citrate; 1 to 8% arginine; 700 to 2000 ppm fluoride; and 2 to 8% alkali phosphate salts selected from sodium phosphate dibasic, potassium phosphate dibasic, dicalcium phosphate dihydrate, tetrasodium pyrophosphate, tetrapotassium pyrophosphate, calcium pyrophosphate, sodium tripolyphosphate, and a combination of two or more thereof.

### Example 6

Oral compositions that comprise arginine are disclosed in WO 2015/094849, which corresponds to US 2016/0338921, which are both incorporated herein by reference. In some embodiments the oral care composition comprises: arginine, in free or salt form; and zinc oxide and zinc citrate. In some embodiments, the arginine is present in an amount of 0.5 weight % to 3 weight %, such as 1 weight % to 2.85 weight %, such as 1.17 weight % to 2.25 weight %, such as 1.4 weight % to 1.6 weight %, such as about 1.5 weight %, based on the total weight of the composition. In some embodiments set out above, the total concentration of zinc salts in the composition is 0.2 weight % to 5 weight %, based on the total weight of the composition. In some embodiments set out above, the molar ratio of arginine to total zinc salts is 0.05:1 to 10:1. In some embodiments set out above, the composition comprises zinc oxide in an amount of 0.5 weight % to 1.5 weight %, such as 1 weight %, and zinc citrate in an amount of 0.25 weight % to 0.75 weight %, such as 0.5 weight %, based on the total weight of the composition. In some embodiments set out above, the weight ratio of zinc oxide to zinc citrate is 1.5:1 to 4.5:1, optionally 1.5:1 to 4:1, 1.7:1 to 2.3:1, 1.9:1 to 2.1:1, or about 2:1.

### Example 7

Oral compositions that comprise arginine are disclosed in WO 2017/003844, which corresponds to US 2018/0021234, which are both incorporated herein by reference. In some embodiments, the oral care composition comprises: arginine, zinc oxide and zinc citrate and a fluoride source. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly salt form. In some embodiments set out above, the ratio of the amount of zinc oxide (by wt %) to zinc citrate (by wt %) is 2:1, 2.5:1, 3:1, 3.5:1 or 4:1, wherein the ratio is by wt. of the overall composition. In some embodiments, the zinc citrate is in an amount of from 0.25 to 1.0 wt % and zinc oxide may be present in an amount of from 0.75 to 1.25 wt % or the zinc citrate is in an amount of about 0.5 wt % and zinc oxide is present in an amount of about 1.0%, based on the total weight of the composition. In some embodiments set out above, the fluoride source is sodium fluoride or sodium monofluorophosphate. In some such embodiments, the sodium fluoride or sodium monofluorophosphate is from 0.1 wt. %-2 wt. % based on the total weight of the composition. In some embodiments, the sodium fluoride or sodium monofluorophosphate is a soluble fluoride salt which provides soluble fluoride in amount of 50 to 25,000 ppm fluoride, such as in an amount of about 1000 ppm-1500 ppm, for example in an amount of about 1450 ppm. In some embodiments the fluoride source is sodium fluoride in an amount about 0.32% by wt, based on the total weight of the composition. In some embodiments, the fluoride source is stannous fluoride. Some embodiments set out above further comprise a preservative selected from: benzyl alcohol, Methylisothizolinone (“MIT”), Sodium bicarbonate, sodium methyl cocoyl taurate (tauranol), lauryl alcohol, and polyphosphate. Some embodiments set out above further comprise benzyl alcohol in an amount of from 0.1-0.8% wt %, or from 0.3-0.5% wt %, or about 0.4 wt % based on the total weight of the composition. In some embodiments, the oral care composition comprises about 1.0% zinc oxide, about 0.5% zinc citrate, about 1.5% L-arginine, about 1450 ppm sodium fluoride, and optionally about benzyl alcohol 0.1 wt. % and/or about 5% small particle silica (e.g., AC43), based on the total weight of the composition. In some embodiments, the oral care composition comprises about 1.0% zinc oxide, about 0.5% zinc citrate, about 5% L-arginine, about 1450 ppm sodium fluoride, and optionally about benzyl alcohol 0.1 wt. % and/or about 5% small particle silica (e.g., AC43), based on the total weight of the composition. In some embodiments set out above, the oral care composition may comprise about 1.0% zinc oxide, about 0.5% zinc citrate, about 1.5% L-arginine, about 0.22%-0.32% sodium fluoride, about 0.5% tetrasodium pyrophosphate, and optionally about benzyl alcohol 0.1 wt. %, based on the total weight of the composition. In some embodiments set out above, the oral care composition may be any of the following oral care compositions selected from the group consisting of: a toothpaste or a dentifrice, a mouthwash or a mouth rinse, a topical oral gel, and a denture cleanser.

### Example 8

Oral compositions that comprise arginine are disclosed in WO 2017/003856, which is incorporated herein by reference. In some embodiments, the oral care composition comprises: arginine in free or salt form, zinc oxide and zinc citrate and a fluoride source comprising stannous fluoride. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly in salt form. In some embodiments set out above, the ratio of the amount of zinc oxide (by wt. %) to zinc citrate (by wt. %) is 2:1, 2.5:1, 3:1, 3.5:1 or 4:1, wherein the ratio is by weight of the overall composition. In some embodiments set out above, the zinc citrate is in an amount of from 0.25 to 1.0 wt. % and zinc oxide may be present in an amount of from 0.75 to 1.25 wt. % or the zinc citrate is in an amount of about 0.5 wt. % and zinc oxide is present in an amount of about 1.0 wt. %, based on the total weight of the composition. In some embodiments set out above, the fluoride source further comprises at least one member selected from the group of: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In some embodiments set out above, the stannous fluoride is present in an amount from 0.1 wt. % to 2 wt. % based on the total weight of the composition. In some embodiments set out above, the stannous fluoride is a soluble fluoride salt which provides soluble fluoride in amount of 50 to 25,000 ppm fluoride, or about 750-7000 ppm, or about 1000-5500 ppm, or about 5000 ppm. In some embodiments, the oral care composition comprises about 1.0% zinc oxide, about 0.5% zinc citrate, about 1.5% L-arginine, about 750-7000 ppm fluoride; and optionally, about 5% small particle silica (e.g., AC43), based on the total weight of the composition. In some embodiments, the oral care composition comprises about 1.0% zinc oxide, about 0.5% zinc citrate, about 750-7000 ppm stannous fluoride; and optionally about 39.2% glycerin based on the total weight of the composition. In some embodiments set out above, the oral care composition may comprise about 1.0% zinc oxide, about 0.5% zinc citrate, about 1.5% L-arginine, stannous fluoride, and optionally about benzyl alcohol 0.1 wt. %, based on the total weight of the composition. In some embodiments set out above, the oral care composition may be any of the following oral compositions selected from the group consisting of: a toothpaste or a dentifrice, a mouthwash or a mouth rinse, a topical oral gel, and a denture cleanser.

### Example 9

A dentifrice composition having the formula of Table A was prepared. The compositions had varying amounts of zinc oxide (varying from 0 to 2 wt %) and of amino acid (varying from 0 to 5 wt %). One example of a dentifrice composition comprised 1 wt % zinc oxide powder, the ZnO being encapsulated, at a 50 wt % loading, into a polymer film, in particular into a polymer film comprising a combination of two different molecular weight HPMC materials, comprising Methocel E5 and Methocel E50. The combined zinc oxide/polymer film comprised 1 wt % ZnO and 1 wt % polymer film, each weight being based on the total weight of the composition. The exemplified dentifrice composition comprised 4.3 wt % L-arginine.

TABLE A

| Ingredient | % w/w | |
|---|---|---|
| Sorbitol | Q.S. | Q.S. |
| Water | 11.994 | 11.994 |
| Silica - Zeodent 105 | 10.000 | 10.000 |
| Silica - Zeodent 114 | 10.00 | 10.00 |
| Polyethylene glycol | 3.00 | 3.00 |
| 600 | | |
| Silica - Zeodent 165 | 2.75 | 2.75 |
| ZnO | 0-2 | 1* |
| Sodium lauryl | 1.500 | 1.500 |
| sulfate | | |
| Cocamidopropyl | 1.250 | 1.250 |
| Betaine | | |
| Flavor | 1.150 | 1.150 |
| Titanium Dioxide | 0.750 | 0.750 |
| Sodium CMC - | 0.065 | 0.065 |
| Type 7MF | | |
| Arginine | 0-5 | 4.3 |
| Sodium Saccharin | 0.270 | 0.270 |
| Sodium Fluoride | 0.243 | 0.243 |

*ZnO in polymer form - 50 wt % loading into polymer film comprising Methocel E5 and Methocel E50 (combined ZnO 1%/polymer - 1%)

Alternative embodiments may be provided which correspond to the composition in Table A in which the arginine is substituted with one or more arginine oligomers selected from R1, R2, R3, R4, R5, R6, R7, R8, R9 and R10. Alternative embodiments may be provided which correspond to the composition in Table A in which the arginine is substituted with a combination of arginine and one or more arginine oligomers selected from R1, R2, R3, R4, R5, R6, R7, R8, R9 and R10.

### Example 10

Test dentifrices comprising 1% and 2% zinc citrate in combination with 5% arginine, 1450 ppm fluoride, and phosphates are prepared as shown in Table B:

TABLE B

| Ingredient | Zinc 1% | Zinc 2% |
|---|---|---|
| PEG600 | 3.00 | 3.00 |
| CMC-7 | 0.65 | 0.65 |
| Xanthan | 0.20 | 0.20 |
| Sorbitol | 28.4 | 27.4 |
| Glycerin | 20.0 | 20.0 |
| Saccharin | 0.30 | 0.30 |
| TSPP | 0.50 | 0.50 |
| cop Phosphate | 0.25 | 0.25 |
| dibasic Phosphate | 3.50 | 3.50 |
| Na Fluoride | 0.32 | 0.32 |
| Water | QS | QS |
| TiO2 | 0.50 | 0.50 |
| Abrasive silica | 8.00 | 8.00 |
| Thickener silica | 8.00 | 8.00 |
| L-Arginine | 5.00 | 5.00 |
| SLS | 1.50 | 1.50 |
| Brighter Flavor K91-5661 | 1.20 | 1.20 |
| Zinc Citrate | 1.00 | 2.00 |

Alternative embodiments may be provided which correspond to the composition in Table B in which the arginine is substituted with one or more arginine oligomers selected from R1, R2, R3, R4, R5, R6, R7, R8, R9 and R10. Alternative embodiments may be provided which correspond to the composition in Table A in which the arginine is substituted with a combination of arginine and one or more arginine oligomers selected from R1, R2, R3, R4, R5, R6, R7, R8, R9 and R10.

### Example 11

Test dentifrices comprising arginine, zinc oxide, zinc citrate and a source of fluoride were prepared as shown in Tables C-G:

TABLE C

| Ingredient | Compound I |
|---|---|
| Humectants | 20.0-25.0 |
| Non-ionic surfactant | 1.0-2.0 |
| Amphoteric surfactant | 3.0-4.0 |
| Flavoring/fragrance/coloring agent | 2.0-3.0 |
| Polymers | 10.0-15.0 |
| pH adjusting agents | 1.5-3.0 |
| Precipitated Calcium Carbonate | 35 |
| Zinc citrate trihydrate | 0.5 |
| Zinc oxide | 1.0 |
| Sodium Fluoride -USP, EP | 0.32 |
| Arginine Bicarbonate | 13.86 |
| Demineralized water | QS |

TABLE D

| Ingredient | Compound A | Compound B | Compound C | Compound D |
|---|---|---|---|---|
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/coloring agent | 2.5-4.0 | 2.5-4.0 | 2.5-4.0 | 2.5-4.0 |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous Precipitated | 16.00 | 21.37 | 17.92 | 7.81 |
| Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride - USP, EP | 0.32 | 0.32 | 0.32 | 0.32 |
| L-Arginine Bicarbonate | 5.0 | 5.0 | 5.0 | 5.0 |
| Demineralized water | QS | QS | QS | QS |

TABLE E

| Ingredient | Compound E | Compound F | Compound G |
|---|---|---|---|
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/coloring agent | 4.0-6.0 | 4.0-6.0 | 4.0-6.0 |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous Precipitated | 17.6 | 8.8 | 22.4 |
| Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine Bicarbonate | 1.5 | 1.5 | 1.5 |
| Demineralized water | QS | QS | QS |

TABLE F

| Ingredient | Compound H | Compound I |
|---|---|---|
| Humectants | 45.0-55.0 | 35.0-45.0 |
| Abrasives | 14.0-16.0 | 9.0-11.0 |
| Anionic surfactant | 1.0-3.0 | 1.0-3.0 |
| Non-ionic surfactant | 0.1-1.0 | — |
| Amphoteric surfactant | 1.0-2.0 | — |
| Flavoring/fragrance/coloring agent | 1.0-3.0 | 2.0-4.0 |
| Polymers | 0.1-2.0 | 3.0-8.0 |
| pH adjusting agents | 0.1-2.0 | 4.0-8.0 |
| Silica Thickener | 5.0 | 5.0-10.0 |
| Benzyl alcohol | 0.1 | — |
| Zinc citrate trihydrate | 0.5 | 0.5 |
| Zinc oxide | 1.0 | 1.0 |
| Sodium Fluoride - USP, EP | 0.32 | 0.32 |
| L-Arginine | 1.5 | 5.0 |
| Demineralized water | QS | QS |

TABLE G

| Ingredient | Compound J | Compound K | Compound L |
|---|---|---|---|
| Humectants | 20.0-50.0 | 20.0-50.0 | 20.0-50.0 |
| Abrasives | 5.0-20.0 | 5.0-20.0 | 5.0-20.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-2.0 | 0.1-2.0 | 0.1-2.0 |
| Flavoring/fragrance/coloring agent | 1.0-5.0 | 1.0-5.0 | 1.0-5.0 |
| Polymers | 0.1-2.0 | 0.1-2.0 | 0.1-2.0 |
| pH adjusting agents | 0.1-2.0 | 0.1-2.0 | 0.1-2.0 |
| Thickener | 6.0 | 6.5 | 7.0 |
| Dental type silica | — | — | 15.0 |
| High cleaning silica | — | 15.0 | — |
| Synthetic Abrasives | 10.0 | — | — |
| Synthetic Amorphous Silica | 5.0 | 5.0 | 5.0 |
| Benzyl alcohol | 0.4 | 0.4 | 0.4 |
| Zinc citrate trihydrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine | 1.5 | 1.5 | 1.5 |
| Demineralized water | QS | QS | QS |

Alternative embodiments may be provided which correspond to the composition in Tables C-G in which the arginine is substituted with one or more arginine oligomers selected from R1, R2, R3, R4, R5, R6, R7, R8, R9 and R10. Alternative embodiments may be provided which correspond to the composition in Table A in which the arginine is substituted with a combination of arginine and one or more arginine oligomers selected from R1, R2, R3, R4, R5, R6, R7, R8, R9 and R10.

### Example 12

Test dentifrices comprising arginine, zinc oxide, zinc citrate and stannous fluoride were prepared as shown in Table H:

TABLE H
Ingredient

| | | | |
|---|---|---|---|
| Humectants | 20.0-60.0 | 20.0-50.0 | 20.0-50.0 |
| Abrasives | 10.0-40.0 | 5.0-20.0 | 5.0-20.0 |
| Anionic surfactant | 1.0-3.0 | 1.0-3.0 | 1.0-3.0 |
| Amphoteric surfactant | 0.5-1.5 | 0.1-2.0 | 0.1-2.0 |
| Flavoring/fragrance/coloring agent | 0.5-5.0 | 1.0-5.0 | 1.0-5.0 |
| Polymers | 1.0-10.0 | 0.1-2.0 | 0.1-2.0 |
| pH adjusting agents | 1.0-10.0 | 0.1-2.0 | 0.1-2.0 |
| Zinc citrate | 0.25-1.0 | 0.5 | 0.5 |
| Zinc oxide | 0.75-1.25 | 1.0 | 1.0 |
| Stannous Fluoride | 0.1-1.0 | 0.32 | 0.32 |
| L-Arginine | 0.1-10.0 | 1.5 | 1.5 |
| Demineralized water | QS | QS | QS |

Alternative embodiments may be provided which correspond to the composition in Table H in which the arginine is substituted with one or more arginine oligomers selected from R1, R2, R3, R4, R5, R6, R7, R8, R9 and R10. Alternative embodiments may be provided which correspond to the composition in Table A in which the arginine is substituted with a combination of arginine and one or more arginine oligomers selected from R1, R2, R3, R4, R5, R6, R7, R8, R9 and R10.

1-32. (canceled)

33. A method of selectively inhibiting co-aggregation of commensal and pathogenic bacteria within an individual's oral cavity comprising the step of: applying 1-50 mg of arginine or an arginine oligomer selected from the group consisting of R1, R2, R3, R4, R5, R6, R7, R8, R9 and R 10, to the oral cavity.

34. The method of claim 33 comprising the step of: applying 1-50 mg of arginine to the oral cavity.

35. The method of claim 33, wherein the commensal bacteria are selected from the group consisting of Actinomyces naeslundii, Streptococcus gordonii, Actinomyces oris and combinations thereof and the pathogenic bacteria are selected from the group consisting of Porphyromonas gingivalis, Fusobacterium nucleatum, Veillonella atypica and combinations thereof.

36. The method of claim 33, wherein 10-50 mg of arginine is applied to the oral cavity.

37. The method of claim 36, wherein 25-50 mg of arginine is applied to the oral cavity.

38. The method of claim 33, wherein 10-40 mg of arginine is applied to the oral cavity.

39. The method of claim 35, wherein commensal bacteria selected from the group consisting of Actinomyces naeslundii, Streptococcus gordonii, Actinomyces oris and combinations thereof and the pathogenic bacteria are selected from the group consisting of Porphyromonas gingivalis, Fusobacterium nucleatum, Veillonella atypica and combinations thereof are present in the individual's oral cavity.

40. The method of claim 33, wherein arginine or arginine oligomer is applied to the oral cavity by using an oral care composition that comprises arginine or arginine oligomer.

41. A method of identifying a compound or composition that enhances the selective inhibition of co-aggregation of commensal and pathogenic bacteria by arginine or arginine oligomer, the method comprising the steps of:
a) performing a bacterial aggregation test assay comprising co-culturing at least one commensal bacteria and at least on pathogenic bacteria in media that comprises arginine or arginine oligomer and a test compound or composition and measuring bacterial aggregation by assigning a score using Visual Co-aggregation Scoring at one or more time points;
b) performing a bacterial aggregation control assay comprising co-culturing at least one commensal bacteria and at least on pathogenic bacteria in media that comprises arginine or arginine oligomer and is free of a test compound or composition and measuring bacterial aggregation by assigning a score using Visual Co-aggregation Scoring at the one or more time points;
c) comparing the score of the bacterial aggregation test assay to the score of the bacterial aggregation control assay;
wherein reduced score in the bacterial aggregation test assay compared to the score in the bacterial aggregation control assay indicates that the test compound or composition enhances inhibition of bacterial aggregation by arginine or arginine oligomer.

42. The method of claim 41, wherein
the commensal oral bacteria is A. naeslundii and pathogenic oral bacteria is P. gingivalis; or
the commensal oral bacteria is S. gordonii and the pathogenic oral bacteria is F. nucleatum; or
the commensal oral bacteria is S. gordonii and the pathogenic oral bacteria V. atypica; or
the commensal oral bacteria is A. oris and the pathogenic oral bacteria is V. atypica; or
the commensal oral bacteria is A. oris and the pathogenic oral bacteria is F. nucleatum, or
the commensal oral bacteria is A. oris and the pathogenic oral bacteria is P. gingivalis.

43. The method of claim 41, wherein the amount of arginine present in the bacterial aggregation test assay is at least 40 mM.

44. The method of claim 41, wherein the amount of arginine present in the bacterial aggregation test assay is at least 86 mM.

45. The method of claim 41, wherein the amount of arginine present in the bacterial aggregation test assay is at least 100 mM.

46. The method of claim 41, wherein the amount of arginine present in the bacterial aggregation test assay is at least 400 mM.

47. A method of identifying a compound or composition that enhances the selective inhibition of biofilm formation of commensal and pathogenic bacteria by arginine or an arginine oligomer, the method comprising the steps of:
a) performing a biofilm formation test assay comprising co-culturing at least one commensal bacteria and at least on pathogenic bacteria in media that comprises arginine or an arginine oligomer and a test compound or composition and measuring biofilm formation at one or more time points;
b) performing a biofilm formation control assay comprising co-culturing at least one commensal bacteria and at least on pathogenic bacteria in media that comprises arginine or an arginine oligomer and is free of a test compound or composition and measuring biofilm formation at the one or more time points;
c) comparing the biofilm formation in the biofilm formation test assay to the biofilm formation in the biofilm formation control assay;
wherein reduced biofilm formation in the biofilm formation test assay compared to the biofilm formation in the biofilm formation control assay indicates that the test compound or composition enhances inhibition of biofilm formation by arginine or an arginine oligomer.
